(19) United States
(12) Patent Application Publication
(10) Pub. No.: US 2022/0059228 A1
(43) Pub. Date: Feb. 24, 2022

(54) SYSTEMS AND METHODS FOR HEALTHCARE INSIGHTS WITH KNOWLEDGE GRAPHS

(71) Applicant: Cambia Health Solutions, Inc., Portland, OR (US)

(72) Inventors: Lin Chen, Bellevue, WA (US); Xiang Li, Bellevue, WA (US); Mao Li, Bothell, WA (US); Sabin Kafle, Bellevue, WA (US); Martin Horn, Sea Tac, WA (US)

(21) Appl. No.: 17/445,571

(22) Filed: Aug. 20, 2021

Related U.S. Application Data
(60) Provisional application No. 63/068,960, filed on Aug. 21, 2020.

(51) Int. Cl. G16H 50/20; G06N 5/04; G06N 5/02; G06F 16/2452; G06F 16/25

(52) U.S. Cl. CPC G16H 50/20; G06N 5/04; G06F 16/254; G06F 16/24522; G06N 5/022

(57) ABSTRACT

Systems and methods for healthcare insights with knowledge graphs are provided. In one example, a method includes constructing, with a processor, a knowledge graph with data from a heterogeneous plurality of data sources, generating, with the processor, healthcare insights from the knowledge graph, and outputting, to a user device for display to a user, a healthcare recommendation based on the healthcare insights. In this way, various types of data may be used to efficiently provide personalized healthcare recommendations for users.

### CROSS REFERENCE TO RELATED APPLICATIONS

The present application claims priority to U.S. Provisional Application No. 63/068,960, entitled “SYSTEMS AND METHODS FOR HEALTHCARE INSIGHTS WITH KNOWLEDGE GRAPHS”, and filed on Aug. 21, 2020. The entire contents of the above-listed application are hereby incorporated by reference for all purposes.

### FIELD

The present description relates generally to deriving healthcare insights from knowledge graphs.

### BACKGROUND AND SUMMARY

In the Information Age, the amount of information stored as digital data is growing exponentially over time. For example, the emergence of electronic health records (EHRs) of patients, as well as an unprecedented amount of drug-related information and disease-related information from pharmaceutical and medical research and development respectively, has resulted in a vast amount of healthcare information being digitally available to researchers and other end-users. Further, increasingly sophisticated techniques have emerged in recent decades to attempt to extract knowledge from the vast amount of digital information. However, such data is typically stored in various structured and unstructured formats across different platforms and systems.

The inventors have recognized the above issues and have devised several approaches to address them. In particular, systems and methods for generating healthcare insights with knowledge graphs are provided. In one embodiment, a method comprises constructing, with a processor, a knowledge graph with data from a heterogeneous plurality of data sources, generating, with the processor, healthcare insights from the knowledge graph, and outputting, to a user device for display to a user, a healthcare recommendation based on the healthcare insights. In this way, various types of data may be used to efficiently provide personalized healthcare recommendations for users.

The above summary is provided to introduce a selection of concepts in a simplified form that are further described below in the Detailed Description. This summary is not intended to identify key features or essential features of the subject matter, nor is it intended to be used to limit the scope of the subject matter. Furthermore, the subject matter is not limited to implementations that solve any or all of the disadvantages noted above or in any part of this disclosure.

### BRIEF DESCRIPTION OF THE FIGURES

FIG. 1 shows a block diagram of an example computing system for deriving healthcare insights from knowledge graphs, according to an embodiment.

FIG. 2 shows a block diagram illustrating an example module architecture for deriving healthcare insights from knowledge graphs, according to an embodiment.

FIG. 3 shows a block diagram illustrating an example scalable architecture for providing healthcare insights derived from knowledge graphs to a user, according to an embodiment.

FIG. 4 shows a block diagram illustrating an example method for deriving healthcare insights personalized for a user, according to an embodiment.

FIG. 5 shows a block diagram illustrating an example knowledge graph for representing healthcare-related entities and relationships, according to an embodiment.

FIG. 6 shows a block diagram illustrating an example method for acquiring and extracting knowledge for a knowledge graph, according to an embodiment.

FIG. 7 shows a high-level flow chart illustrating an example method for deriving healthcare insights from a knowledge graph, according to an embodiment.

### DETAILED DESCRIPTION

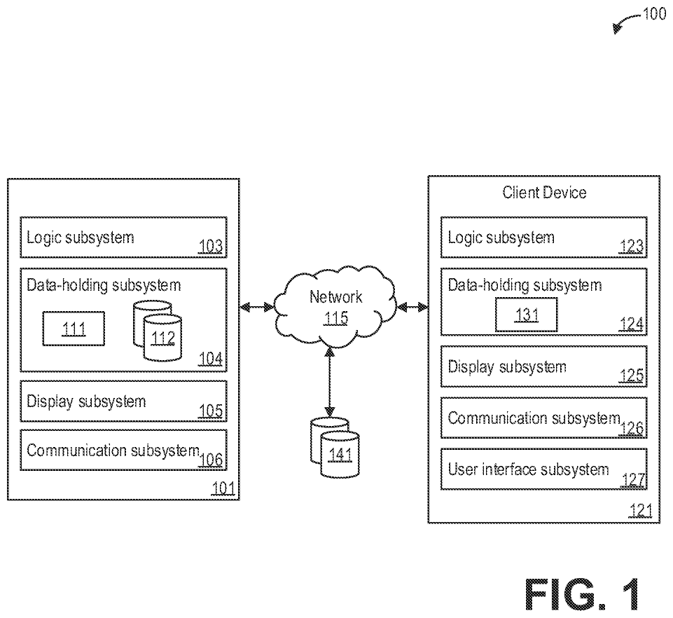

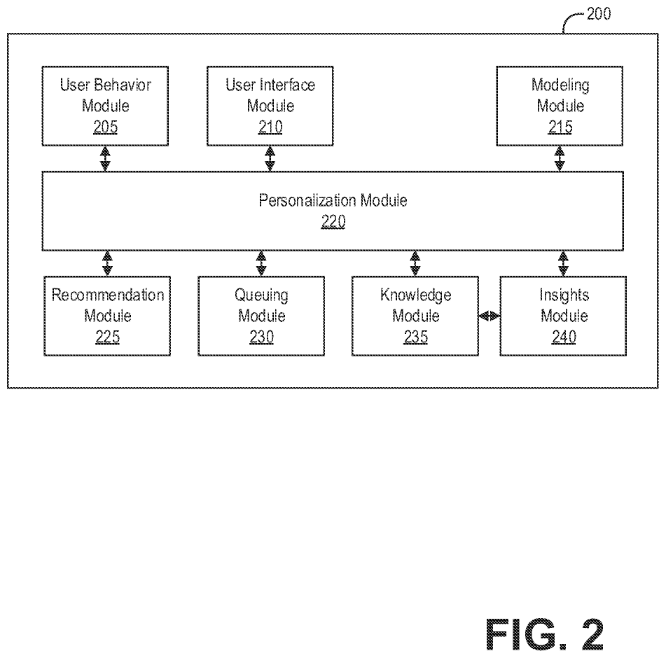

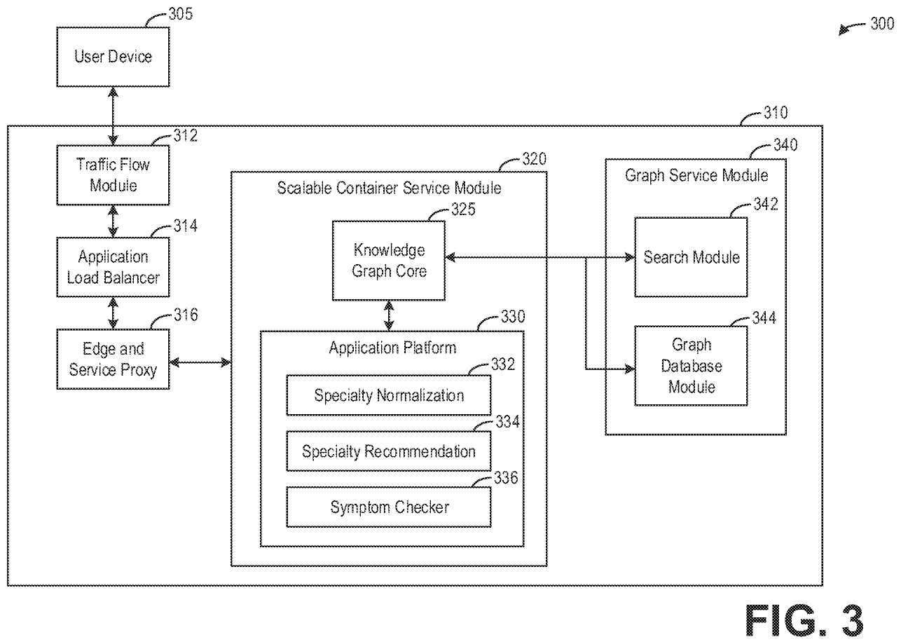

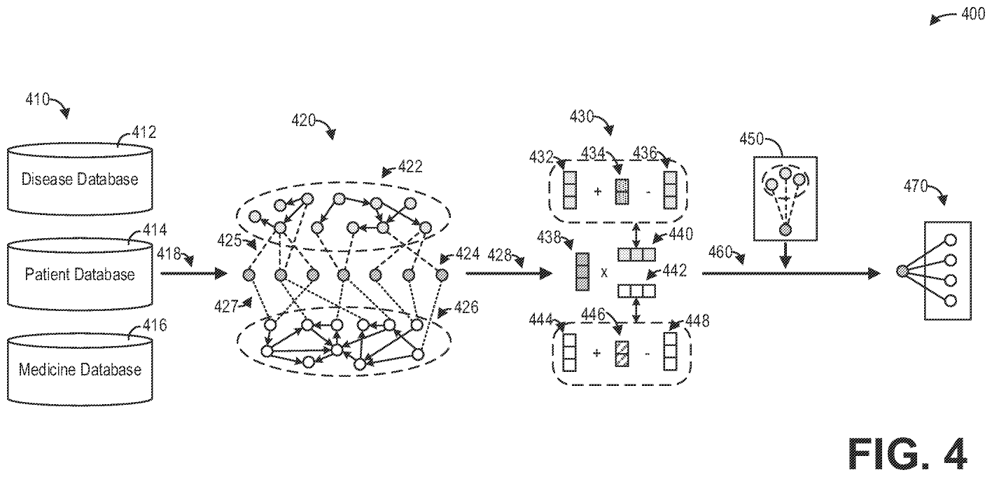

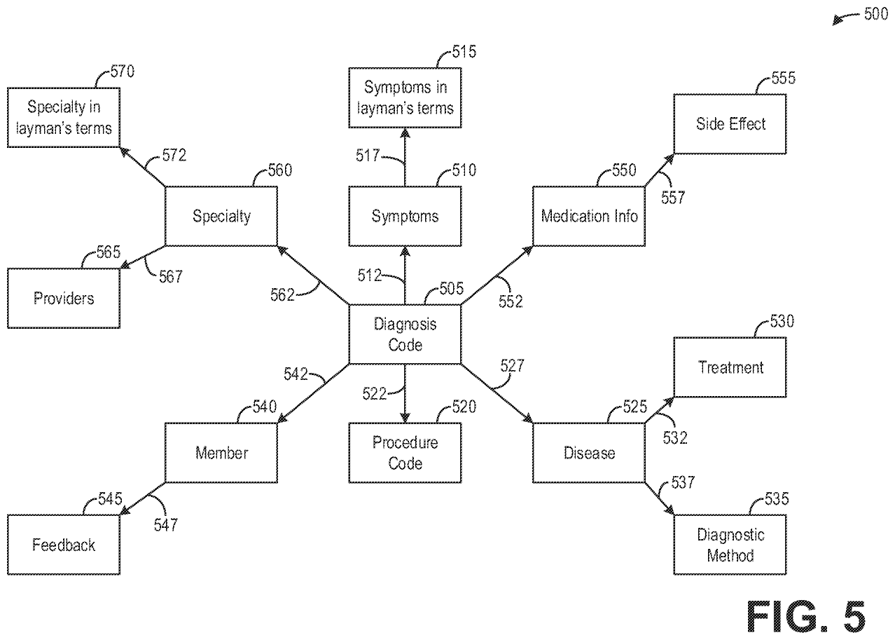

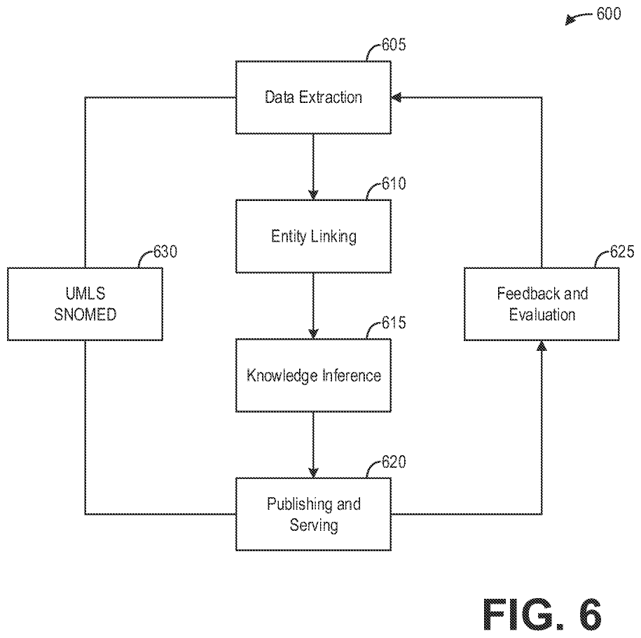

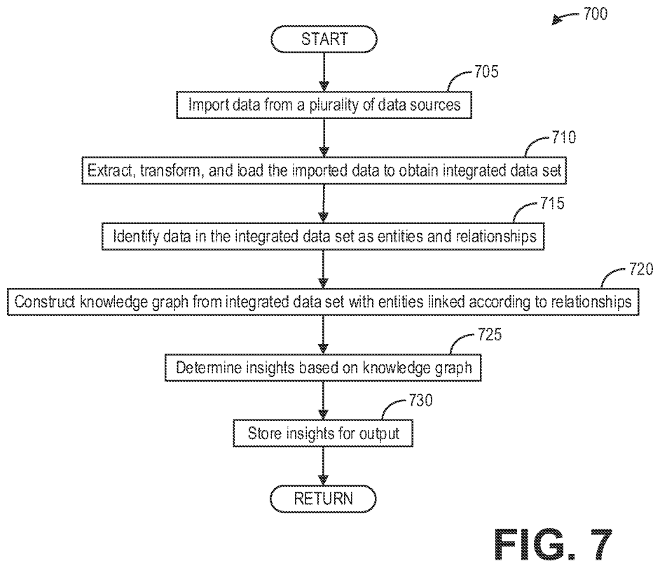

The present description relates to systems and methods for healthcare insights with knowledge graphs. In particular, systems and methods are provided for constructing a knowledge graph from data obtained from a plurality of data sources, and deriving healthcare insights and inferences from the knowledge graph. A knowledge graph comprises a directed heterogeneous graph-structured data model that encodes a network of entities and their relationships. Healthcare insights automatically derived from such knowledge graphs may be used by healthcare providers, such as nurses, to enhance the provision of care, or may be directly shared with patients or other users. A computing environment, such as the computer environment or system depicted in FIG. 1, may include a healthcare knowledge system which consolidates data from a disparate and heterogeneous plurality of data sources into a knowledge graph, and derives new healthcare insights from the knowledge graph. Such a healthcare knowledge system, as depicted in FIG. 2, may include modules configured to provide artificially-intelligent applications powered by the knowledge graph. Further, the healthcare knowledge system, as depicted in FIG. 3, may be implemented with a highly scalable cloud computing architecture to accommodate an increasing amount of data, an increasing number of users, and an increasing number of applications. The knowledge graph may be constructed, as depicted in FIG. 4, from a plurality of data sources including disease databases, patient databases, and medicine databases in order to provide, for example, healthcare recommendations for patients. The knowledge graph, for example as depicted in FIG. 5, may systematically organize a plurality of different concepts and their relationships, represented as entities and edges respectively in the knowledge graph. The knowledge graph may be iteratively updated through a bootstrapping process, as depicted in FIG. 6, to further incorporate additional knowledge over time, as well as to improve responsive to user feedback and behavior. An example method for constructing a knowledge graph and deriving insights from the knowledge graph is depicted in FIG. 7.

FIG. 1 illustrates an example computing environment 100 in accordance with the current disclosure. In particular, computing environment 100 includes a server 101, a plurality of user devices or client systems including at least one client device 121, and a network 115. However, not all of the components illustrated may be required to practice the invention. Variations in the arrangement and type of the components may be made without departing from the spirit or scope of the invention.

Server 101 may be a computing device configured to construct knowledge graphs from data obtained from heterogeneous healthcare data sources, generates healthcare insights and inferences from the knowledge graph, and uses such insights for improving various applications such as recommendation systems and search engines. In different embodiments, server 101 may take the form of a mainframe computer, server computer, desktop computer, laptop computer, tablet computer, network computing device, mobile computing device, mobile communication device, and so on.

Server 101 includes a logic subsystem 103 and a data-holding subsystem 104. Server 101 may optionally include a display subsystem 105, communication subsystem 106, and/or other components not shown in FIG. 1. For example, server 101 may also optionally include user input devices such as keyboards, mice, game controllers, cameras, microphones, and/or touch screens.

Logic subsystem 103 may include one or more physical devices configured to execute one or more instructions. For example, logic subsystem 103 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 103 may include one or more processors that are configured to execute software instructions. Additionally or alternatively, the logic subsystem 103 may include one or more hardware or firmware logic machines configured to execute hardware or firmware instructions. Processors of the logic subsystem 103 may be single or multi-core, and the programs executed thereon may be configured for parallel or distributed processing. The logic subsystem 103 may optionally include individual components that are distributed throughout two or more devices, which may be remotely located and/or configured for coordinated processing. One or more aspects of the logic subsystem 103 may be virtualized and executed by remotely accessible networked computing devices configured in a cloud computing configuration.

Data-holding subsystem 104 may include one or more physical, non-transitory devices configured to hold data and/or instructions executable by the logic subsystem 103 to implement the herein described methods and processes. When such methods and processes are implemented, the state of data-holding subsystem 104 may be transformed (for example, to hold different data).

In one example, the server 101 includes a healthcare knowledge system 111 configured as executable instructions in the data-holding subsystem 104. The healthcare knowledge system 111 may import and/or extract a plurality of data from a plurality of data sources 141, transform and structure the data as a knowledge graph, and generate insights from the knowledge graph. To effectively predict or assess the potential risk of a member, for example, the plurality of models may process one or more healthcare claims associated with the member. To that end, one or more healthcare databases 112 comprising one or more knowledge graphs storing aggregated data from a plurality of data sources 141 may be stored in the data-holding subsystem 104 and accessible to the healthcare knowledge system 111. That is, the one or more healthcare databases 112 comprise graph databases rather than relational databases. The one or more healthcare databases 112 may include healthcare data and other data stored locally or remotely, for example in one or more databases or data sources 141 communicatively coupled to the server 101 via the network 115.

Data-holding subsystem 104 may include removable media and/or built-in devices. Data-holding subsystem 104 may include optical memory (for example, CD, DVD, HD-DVD, Blu-Ray Disc, etc.), and/or magnetic memory devices (for example, hard drive disk, floppy disk drive, tape drive, MRAM, etc.), and the like. Data-holding subsystem 104 may include devices with one or more of the following characteristics: volatile, nonvolatile, dynamic, static, read/write, read-only, random access, sequential access, location addressable, file addressable, and content addressable. In some embodiments, logic subsystem 103 and data-holding subsystem 104 may be integrated into one or more common devices, such as an application-specific integrated circuit or a system on a chip.

It is to be appreciated that data-holding subsystem 104 includes one or more physical, non-transitory devices. In contrast, in some embodiments aspects of the instructions described herein may be propagated in a transitory fashion by a pure signal (for example, an electromagnetic signal) that is not held by a physical device for at least a finite duration. Furthermore, data and/or other forms of information pertaining to the present disclosure may be propagated by a pure signal.

When included, display subsystem 105 may be used to present a visual representation of data held by data-holding subsystem 104. As the herein described methods and processes change the data held by the data-holding subsystem 104, and thus transform the state of the data-holding subsystem 104, the state of display subsystem 105 may likewise be transformed to visually represent changes in the underlying data. Display subsystem 105 may include one or more display devices utilizing virtually any type of technology. Such display devices may be combined with logic subsystem 103 and/or data-holding subsystem 104 in a shared enclosure, or such display devices may be peripheral display devices.

When included, communication subsystem 106 may be configured to communicatively couple server 101 with one or more other computing devices, such as client device 121. Communication subsystem 106 may include wired and/or wireless communication devices compatible with one or more different communication protocols. As non-limiting examples, communication subsystem 106 may be configured for communication via a wireless telephone network, a wireless local area network, a wired local area network, a wireless wide area network, a wired wide area network, etc. In some embodiments, communication subsystem 106 may allow server 101 to send and/or receive messages to and/or from other devices via a network such as the public Internet. For example, communication subsystem 106 may communicatively couple server 101 with client device 121 via network 115. In some examples, network 115 may be the public Internet. In other examples, network 115 may be regarded as a private network connection and may include, for example, a virtual private network or an encryption or other security mechanism employed over the public Internet.

Further, the server 101 provides a network service that is accessible to a plurality of users through a plurality of client systems such as the client device 121 communicatively coupled to the server 101 via the network 115. As such, computing environment 100 may include one or more devices operated by users, such as client device 121. User device 121 may be any computing device configured to access a network such as network 115, including but not limited to a personal desktop computer, a laptop, a smartphone, a tablet, and the like. While one client device 121 is shown, it should be appreciated that any number of user devices may be communicatively coupled to the server 101 via the network 115.

Client device 121 includes a logic subsystem 123 and a data-holding subsystem 124. Client device 121 may optionally include a display subsystem 125, communication subsystem 126, a user interface subsystem 127, and/or other components not shown in FIG. 1.

Logic subsystem 123 may include one or more physical devices configured to execute one or more instructions. For example, logic subsystem 123 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 123 may include one or more processors that are configured to execute software instructions. Additionally or alternatively, the logic subsystem 123 may include one or more hardware or firmware logic machines configured to execute hardware or firmware instructions. Processors of the logic subsystem 123 may be single or multi-core, and the programs executed thereon may be configured for parallel or distributed processing. The logic subsystem 123 may optionally include individual components that are distributed throughout two or more devices, which may be remotely located and/or configured for coordinated processing. One or more aspects of the logic subsystem 123 may be virtualized and executed by remotely accessible networking computing devices configured in a cloud computing configuration.

Data-holding subsystem 124 may include one or more physical, non-transitory devices configured to hold data and/or instructions executable by the logic subsystem 123 to implement the herein described methods and processes. When such methods and processes are implemented, the state of data-holding subsystem 124 may be transformed (for example, to hold different data).

Data-holding subsystem 124 may include removable media and/or built-in devices. Data-holding subsystem 124 may include optical memory (for example, CD, DVD, HD-DVD, Blu-Ray Disc, etc.), and/or magnetic memory devices (for example, hard drive disk, floppy disk drive, tape drive, MRAM, etc.), and the like. Data-holding subsystem 124 may include devices with one or more of the following characteristics: volatile, nonvolatile, dynamic, static, read/write, read-only, random access, sequential access, location addressable, file addressable, and content addressable. In some embodiments, logic subsystem 123 and data-holding subsystem 124 may be integrated into one or more common devices, such as an application-specific integrated circuit or a system on a chip.

When included, display subsystem 125 may be used to present a visual representation of data held by data-holding subsystem 124. As the herein described methods and processes change the data held by the data-holding subsystem 124, and thus transform the state of the data-holding subsystem 124, the state of display subsystem 125 may likewise be transformed to visually represent changes in the underlying data. Display subsystem 125 may include one or more display devices utilizing virtually any type of technology. Such display devices may be combined with logic subsystem 123 and/or data-holding subsystem 124 in a shared enclosure, or such display devices may be peripheral display devices.

In one example, the client device 121 may include executable instructions 131 in the data-holding subsystem 124 that when executed by the logic subsystem 123 cause the logic subsystem 123 to perform various actions as described further herein. As one example, the client device 121 may be configured, via the instructions 131, to provide data regarding a patient to the server 101, receive one or more healthcare recommendations from the server 101 generated based on the data regarding the patient and a knowledge graph stored at the server 101, and display the one or more healthcare recommendations via a graphical user interface on the display subsystem 125 to a user such as a healthcare provider or the patient. The client device 121 may be further configured to receive feedback regarding the one or more healthcare recommendations via the user interface subsystem 127.

When included, communication subsystem 126 may be configured to communicatively couple client device 121 with one or more other computing devices, such as server 101. Communication subsystem 126 may include wired and/or wireless communication devices compatible with one or more different communication protocols. As non-limiting examples, communication subsystem 126 may be configured for communication via a wireless telephone network, a wireless local area network, a wired local area network, a wireless wide area network, a wired wide area network, etc. In some embodiments, communication subsystem 126 may allow client device 101 to send and/or receive messages to and/or from other devices, such as server 101, via a network 115 such as the public Internet.

Client device 121 may further include a user input subsystem 127 comprising user input devices such as keyboards, mice, game controllers, cameras, microphones, and/or touch screens. A user of client device 121 may input feedback regarding a healthcare recommendation, for example, via user input subsystem 127. As discussed further herein, client device 121 may stream, via communication subsystem 126, user input received via the user input subsystem 127 to the server 101 over the network 115. In this way, the server 101 may update one or more models for generating healthcare recommendations and/or one or more knowledge graphs based on the user feedback.

Thus server 101 and client device 121 may each represent computing devices which may generally include any device that is configured to perform computation and that is capable of sending and receiving data communications by way of one or more wired and/or wireless communication interfaces. Such devices may be configured to communicate using any of a variety of network protocols. For example, client device 121 may be configured to execute a browser application that employs HTTP to request information from server 101 and then displays the retrieved information to a user on a display such as the display subsystem 125.

FIG. 2 shows an overview of an exemplary arrangement of software modules for a healthcare knowledge system 200 for assembling healthcare data and deriving healthcare insights for users such as healthcare providers, patients, and/or other entities. The healthcare knowledge system 200 may be implemented, as an illustrative example, as instructions 111 in the data-holding subsystem 104 of a server 101. The healthcare knowledge system 200 comprises a plurality of modules, including but not limited to a user behavior module 205, a user interface module 210, a modeling module 215, a personalization module 220, a recommendation module 225, a queuing module 230, a knowledge module 235, and an insights module 240. The modules depicted are exemplary, and in some examples the healthcare knowledge system 200 may include different combinations of modules including or not including the modules depicted in FIG. 2 as described further herein. As depicted, the personalization module 220 facilitates much of the interactions between other modules of the healthcare knowledge system 200.

The user behavior module 205 is configured to receive and aggregate user feedback regarding recommendations provided by the healthcare knowledge system 200. For example, the user behavior module 205 may receive indications of user engagement with a healthcare recommendation, such as reviewing the recommendation, accepting the recommendation, rejecting the recommendation, and so on. Such user behavior may be encoded in the knowledge graph(s) described herein as user feedback, which in turn may enable further refinement of the knowledge graph and subsequent healthcare recommendations. For example, based on user behavior, recommendations may be ranked and filtered.

The user interface module 210 is configured to generate a user interface for transmission to a user device, such as a client device 121, which may be displayed as a graphical user device via a display subsystem 125, for example. The user interface module 210 may further receive data from the client device 121, for example, and provide the received data to one or more modules of the healthcare knowledge system 200. In this way, the user interface module 210 manages interactions between a user and the healthcare knowledge system 200.

The modeling module 215 is configured to construct and train machine learning model(s), for example, to provide artificially intelligent services to users. For example, the modeling module 215 may include one or more machine learning models trained as a chatbot, which may utilize the knowledge graph of the knowledge module 235 and/or insights of the insights module 240, for example, to automatically converse via text with a user. Each model of the modeling module 215 may comprise a machine learning model, as an illustrative and non-limiting example. For example, one or more of the models may comprise a machine learning model trained via supervised or unsupervised learning. To that end, a model of the modeling module 215 may comprise, as a non-limiting and illustrative example, one or more of an artificial neural network, a linear regression model, a logistic regression model, a linear discriminant analysis model, a classification or regression tree model, a naïve Bayes model, a k-nearest neighbors model, a learning vector quantization model, a support vector machine, a random forest model, a boosting model and so on. The models in particular may comprise different types of machine learning models.

The personalization module 220 is configured to coordinate personalized output for a given user based on data from the insights module 240, the user behavior module 205, the modeling module 215, the knowledge module 235, the queuing module 230, and the recommendation module 225. For example, the personalization module 220 facilitates interactions between modules of the healthcare knowledge system 200 to provide output that is personalized to a user as well as personalized for a given patient.

The recommendation module 225 is configured to generate recommendations such as healthcare recommendations for users based on the insights determined by the insights module 240, for example, from the knowledge graph constructed and updated by the knowledge module 235. A healthcare recommendation may comprise, as an illustrative and non-limiting example, a recommendation for a medicine to prescribe a patient based on one or more symptoms experienced by the patient.

The queuing module 230 is configured to manage integrated data updates into the knowledge graph managed by the knowledge module 235, for example in a data update queue, as well as to manage the output of recommendations determined based on insights, for example in a recommendation queue. For example, while an initial knowledge graph may be constructed from initial data sources, the initial knowledge graph may be regularly updated by incrementally integrated or consolidating further and additional datasets into the knowledge graph. The queuing module 230 may therefore organize and queue updates to the knowledge graph.

The knowledge module 235 is configured to aggregate data from a plurality of data sources into a knowledge graph. For example, the knowledge module 235 may import data from a plurality of data sources, perform extraction, transformation, and loading (ETL) of the data, and represent the data as entities and relationships in a knowledge graph. An example knowledge graph is described further herein with regard to FIG. 5, and example methods for constructing a knowledge graph are described further herein with regard to FIGS. 6 and 7.

The insights module 240 is configured to generate healthcare insights from a knowledge graph. To that end, the insights module 240 may be configured to generate knowledge graph embeddings from one or more knowledge graphs. Knowledge graph embeddings comprise lower-level representations of the entities and relationships depicted in a knowledge graph. To that end, the insights module 240 may represent each entity and relation of a given knowledge graph with a vector. For example, given a knowledge graph, the insights module 240 may generate a vector KG=(E, R, W), where E is a set of entities, R is a set of edges between entities, and W is a set of edge weights. Such a vector preserves the knowledge graph structure with a reduced dimensionality relative to the graph. The insights module 240 may use one or more knowledge graph embedding models to generate the embeddings from the knowledge graphs. For example, the insights module 240 may be configured to use a translation-based embedding model, a matrix factorization model, or a neural network model. The insights module 240 may then infer new knowledge or derive new insights using the knowledge graph embeddings of the knowledge graph, as described further herein.

FIG. 3 shows a block diagram illustrating an example scalable cloud-computing architecture 300 for a healthcare knowledge system 310 configured to provide healthcare insights derived from knowledge graphs to a user of a user device 305, according to an embodiment. The user device 305 may comprise the client device 121 depicted in FIG. 1 and described hereinabove, for example, while the healthcare knowledge system 310 may comprise the healthcare knowledge system 200 described hereinabove. The scalable cloud-computing architecture 300 in particular depicts a plurality of modules to enable an elastic and scalable platform based on a knowledge graph, such that the platform may be accessed by an arbitrarily large number of users.

To that end, the healthcare knowledge system 310 includes a traffic flow module 312 configured to effectively connect user requests to infrastructure of the healthcare knowledge system 310. For example, while a user may attempt to access the healthcare knowledge system 310 via a set domain name (e.g., www.example.com), the traffic flow module 312 dynamically routes the user to application endpoints.

For example, user requests from the user device 305 may be dynamically routed via the traffic flow module 312 to an application load balancer 314. The application load balancer 314 distributes the incoming traffic across multiple targets, such as multiple elastic compute cloud (EC2) instances. For example, the application load balancer 314 may distribute the traffic to an edge and service proxy 316, which in turn directs the traffic to a scalable container service module 320.

The scalable container service module 320 enables the execution of modules contained therein in a managed cluster of EC2 instances. To that end, the scalable container service module 320 may include a knowledge graph core 325 and an application platform 330. The user of the user device 305 thus interacts with the application platform 330, which may provide services such as a specialty normalization 332, a specialty recommendation 334, a symptom checker 336, and so on, which are powered by the knowledge graph core 325. The knowledge graph core 325 may comprise, for example, the insights module 240 and knowledge module 235 as described hereinabove. To provide dynamic knowledge graph functionality for the application platform 330, a graph service module 340 includes a search module 342 and a graph database module 344. The search module 342 may enable search via elasticsearch, for example, while the graph database module 344 provides graph data storage and operation functionality.

FIG. 4 shows a block diagram illustrating an example method 400 for deriving healthcare insights personalized for a user, according to an embodiment. In particular, method 400 relates to constructing a knowledge graph from a plurality of data sources, deriving healthcare insights from the knowledge graph, and providing personalized healthcare recommendations to users based on the healthcare insights.

A plurality of databases or data sources 410 includes, for example, one or more disease databases 412, one or more patient databases 414, and one or more medicine databases 416. For example, the one or more disease databases 412 may comprise a dataset of an ICD-9 ontology which maps diagnostic codes (e.g., ICD-9 codes) to related terms. The one or more patient databases 414 may include, for example, a database such as MIMIC-III (“Medical Information Mart for Intensive Care”) which comprises a large, single-center database including information relating to patients admitted to critical care units at a large tertiary care hospital. Such a database may include de-identified data including but not limited to vital signs, medications, laboratory measurements, observations and notes charted by care providers, fluid balance, procedure codes, diagnostic codes, imaging reports, hospital length of stay, survival data, and so on. The one or more medicine databases 416 may include, for example, the DrugBank database which includes detailed drug (i.e., chemical, pharmacological, and pharmaceutical) data with comprehensive drug target (i.e., sequence, structure, and pathway) information.

Method 400 thus constructs 418 a heterogeneous knowledge graph 420 from the data stored in the plurality of databases 410. The knowledge graph 420, as depicted, includes a plurality of entities and edges connecting the entities. For example, the knowledge graph 420 includes a plurality of disease entities 422 with a plurality of directed disease edges indicating relationships between the disease entities 422, a plurality of patient entities 424, a plurality of patient-disease edges 425 indicating relationships between the patient entities 424 and the disease entities 422, a plurality of medicine entities 426 with a plurality of directed medicine edges indicating relationships between the medicine entities 426, and a plurality of patient-disease edges 427 indicating relationships between the patient entities 424 and the medicine entities 426.

Method 400 then trains knowledge graph embeddings 430 with the knowledge graph 420 based on a knowledge graph embedding model, such as a translation-based embedding model. The knowledge graph embeddings 430 comprise jointly-learned embeddings, including, as depicted: disease embeddings 432, 436, and 440; patient embeddings 438; medicine embeddings 442, 444, and 448; and relation embeddings 434 and 446. As discussed hereinabove, the knowledge graph embeddings 430 comprise a lower-dimensional representation of the knowledge graph 420.

Method 400 receives new patient data 450 comprising, for example, a patient entity linked to one or more disease entities as depicted. Based on the new patient data 450, method 400 then generates 460 a medicine recommendation 470, for example, based on the knowledge graph embeddings 430 and thus on the knowledge graph 420. For example, method 400 identifies one or more medicine entities linked to a patient entity which is in turn linked to the same set of disease entities as indicated in the new patient data 450, and returns the one or more medicine entities as the medicine recommendation 470, as depicted.

Although the knowledge graph 420 described hereinabove includes disease, patient, and medicine entities, the knowledge graphs of the present disclosure may include additional information. As an illustrative example, FIG. 5 shows a block diagram illustrating an example knowledge graph 500 for representing healthcare-related entities and relationships, according to an embodiment. In particular, the knowledge graph 500 depicts example entities or nodes and edges for a given diagnosis code 505 or diagnosis code entity 505. The diagnosis code 505 may be associated with one or more symptoms entities 510 via one or more diagnosis code-symptom edges 512. The symptoms of the symptoms entity 510 may further be associated with an entity 515 indicating the symptoms in layman's terms via a symptom-layman edge 517. In this way, the knowledge graph 500 may be used to associate the symptoms 510 in plain language or layman's terms 515 with other entities of the knowledge graph 500, thereby improving search results and artificial intelligent chatbots powered by the knowledge graph 500, as illustrative examples.

The knowledge graph 500 further includes one or more procedure code entities 520 associated with the diagnosis code entity 505 via one or more diagnosis code-procedure code edges 522. Similarly, the diagnosis code entity 505 is associated with a disease entity 525 for a disease via a diagnosis code-disease edge 527. The disease entity 525 is further linked to a treatment entity 530 for a treatment of the disease 525 via a disease-treatment edge 532, and a diagnostic method entity 535 for a diagnostic method of diagnosing the disease 525 via a disease-diagnostic method edge 537.

The knowledge graph 500 further includes a member entity 540 associated with the diagnosis code 505 via a diagnosis code-member edge 542. The member 540 is further linked to a feedback entity 545 via a member-feedback edge 547, wherein the feedback entity 545 includes user feedback for the member 540. In this way, members 540 or patients may be linked, via the knowledge graph 500, to diagnosis codes 505 and thus to symptoms 510, diseases 525, and so on.

The knowledge graph 500 further includes a medication information entity 550 associated with the diagnosis code 505 via a diagnosis code-medication edge 552. The medication information 550 may in turn be associated with one or more side effect entities 555 via a medication-side effect edge 557.

Similarly, the diagnosis code 505 is associated with at least one specialty entity 560 via a diagnosis code-specialty edge 562. The specialty or specialty entity 560 indicates a healthcare specialty associated with the diagnosis code 505. Further, one or more providers 565 are associated with the specialty 560 via one or more corresponding specialty-provider edge(s) 567. The specialty 560 is further associated with an entity 570 indicating the specialty in layman's terms via a specialty-laymen edge 572.

Thus, by organizing data as depicted in the knowledge graph 500, the different entities such as symptoms, specialties, providers, diseases, treatments, diagnostic methods, medication, side effects, patients or members, and even user feedback may be associated.

FIG. 6 shows a block diagram illustrating an example method 600 for acquiring and extracting knowledge for a knowledge graph, according to an embodiment. Method 600 may be carried out by a healthcare knowledge system, such as the healthcare knowledge system 200 described hereinabove with regard to FIG. 2.

At 605, method 600 performs data extraction. For example, method 600 performs web crawling of biomedical literature, for example in a network-connected archive or database such as PubMed. At 610, method 600 performs entity linking. For example, method 600 may use Unified Medical Language System (UMLS) identifiers (IDs) with natural language processing techniques, including neural language models trained for medical entity linking (e.g., MedLinker), to map text in the extracted data to entities in the knowledge base or knowledge graph. To that end, entity linking includes constructing a knowledge graph and/or updating a knowledge graph with the extracted data, for example to connect the identified entities to other entities of the knowledge graph. Entity linking may be performed via a trained neural network model, for example, and/or natural language processing (NLP) models.

At 615, method 600 performs knowledge inference. To that end, method 600 performs relationship extraction, for example by detecting and classifying relationships between entities. Further, method 600 generates new knowledge candidates based on the extracted relationships, for example by using a rule-based technique for generating the new candidates and then evaluating whether the candidates match entities in the knowledge graph. For example, a score may be computed for each new candidate, and candidates may be added and linked to the knowledge graph if the score is above a threshold. A ranking and filtering strategy may be used, for example to take the domain relevance and trustworthiness of the origin of the data into account.

At 620, method 600 performs publishing and serving of the knowledge graph. To that end, method 600 trains knowledge graph embedding based on the updated knowledge graph, and further performs embedding-based validation. The knowledge graph embedding may be used for generating new recommendations based on new patient data, for example as described hereinabove with regard to FIG. 4.

Method 600 then obtains feedback and evaluation at 625, for example by joint learning with text, and bootstrapping the extraction pipeline. That is, the extraction pipeline comprising the method 600 may be a stepwise or iterative learning process in which the knowledge graph published and served at 620 is treated as the initial knowledge to be updated at 605.

Method 600 may rely on medical or healthcare-related ontologies, such as UMLS and Systematized Nomenclature of Medicine (SNOMED), as depicted at 630 for example, to perform both the data extraction at 605 as well as the publishing and serving at 625.

FIG. 7 shows a high-level flow chart illustrating an example method 700 for deriving healthcare insights from a knowledge graph, according to an embodiment. In particular, method 700 relates to constructing a knowledge graph from a plurality of data sources and deriving healthcare insights from the knowledge graph, which in turn may be used to provide healthcare recommendations to users. Method 700 is described with regard to the systems and components of FIGS. 1-6, though it should be appreciated that the method 700 may be implemented with other systems and components without departing from the scope of the present disclosure. Method 700 may be implemented as executable instructions in non-transitory memory, such as the data-holding subsystem 104, and may be executed by a processor, such as the logic subsystem 103.

Method 700 begins at 705. At 705, method 700 imports data from a plurality of data sources. For example, the server 101 may import data from the plurality of data sources 141 into the data-holding subsystem 104. Further, at 710, method 700 extracts, transforms, and loads the imported data to obtain an integrated data set. That is, method 700 extracts the imported data, converts or transforms the extracted data into a different format or structure, and stores or loads the transformed data into a graph structure. In this way, the extract, transform, load (ETL) process converts the data that may have been previously stored in relational database structures or other files (e.g., raw text) into a graph format. Further, the transformation step may include conversion of the relations encoded in the data in relational database structures into entities and edges or links therebetween.

At 715, method 700 identifies data in the integrated data set as entities and relationships. As mentioned hereinabove, during the ETL process some of the data is already transformed into entities and relationships. Method 700 further identifies additional entities and links therebetween, for example in order to connect data from disparate data sources. For example, a first data source may include patient or member data with diagnosis codes, a second data source may include provider data and specialties, and a third data source may include medication information. As an illustrative example, the data sources may include specialty layman's terms, provider specialty, provider types, diagnosis codes, symptoms data, Medicare specialty, drug data, disease information, treatment data, member data from medical claims, user interaction data, and so on. While the ETL process may identify entities and edges for data within each data source, at 720 method 700 may identify connections between such data sources in order to link the entities as depicted in FIG. 5. Then, at 720, method 700 constructs a knowledge graph from the integrated data set with the entities linked according to the relationships. That is, method 700 integrates the identified entities and relationships or edges into a single knowledge graph structure.

At 725, method 700 determines insights based on the knowledge graph. For example, method 700 may use a trained machine learning model (e.g., trained and stored in the modeling module 215) to automatically identify new connections within the knowledge graph. For example, while a given member or patient may be linked to a diagnosis code and a particular medication in the knowledge graph because the patient is prescribed the particular medication, the knowledge graph may indicate that a large plurality of similar patients (i.e., other patients linked to similar symptoms, diagnosis code, disease, and so on) are prescribed a different medication with a lower cost. Method 700 may thus identify the potential for prescribing the medication as an alternative to the patient as an insight. Similarly, healthcare providers may be identified with certain symptoms and diseases, for example, thereby providing a certain healthcare insight that may be useful for users searching for providers who treat a given disease.

At 730, method 700 stores the insights for output. Such healthcare insights may be stored separately, for example in the insights module 240, or may be stored in the knowledge graph itself. The stored insights and the knowledge graph may be used to serve personalized recommendations in various applications and systems, for example in personalized provider searches, personalized recommendations based on symptoms, healthcare decision support systems, and so on. Method 700 then returns.

A technical effect of the present disclosure includes the automatic generation of a recommendation based on disparate data from a plurality of data sources. Another technical effect of the present disclosure includes the output, such as the display, of a healthcare recommendation automatically generated based on a knowledge graph containing data from a plurality of data sources. Yet another technical effect includes the transformation of raw data from a plurality of data sources into a unified knowledge graph that links information from different data sources of the plurality of data sources.

In one embodiment, a method comprises constructing, with a processor, a knowledge graph with data from a heterogeneous plurality of data sources, generating, with the processor, healthcare insights from the knowledge graph, and outputting, to a user device for display to a user, a healthcare recommendation based on the healthcare insights.

In a first example of the method, the heterogeneous plurality of data sources includes a provider data source, a patient data source, a medicine data source, a disease database, and at least one medical ontology data source. In a second example of the method optionally including the first example, the method further comprises training knowledge graph embeddings based on the knowledge graph, receiving new patient data, and generating the healthcare recommendation based on the knowledge graph embeddings and the new patient data. In a third example of the method optionally including one or more of the first and second examples, the method further comprises updating the knowledge graph with user feedback and user behavior regarding the healthcare recommendations, and ranking subsequent healthcare recommendations based on the updated knowledge graph. In a fourth example of the method optionally including one or more of the first through third examples, the healthcare insights include one or more of newly-identified edges between entities in the knowledge graph, including one or more of a relationship between a patient and a medication, a relationship between a symptom and a diagnostic code, and a relationship between a healthcare provider and a disease. In a fifth example of the method optionally including one or more of the first through fourth examples, the method further comprises updating the knowledge graph to include entities relating plain language terminology to medical terminology, and performing a search responsive to a user query including the plain language terminology for one or more of healthcare providers and symptoms with the knowledge graph.

In another embodiment, a computer-readable storage medium includes an executable program stored thereon, the program configured to cause a computer processor to: construct a knowledge graph with data from a heterogeneous plurality of data sources; generate healthcare insights from the knowledge graph; and output, to a user device for display to a user, a healthcare recommendation based on the healthcare insights.

In a first example of the computer-readable storage medium, the heterogeneous plurality of data sources includes a provider data source, a patient data source, a medicine data source, a disease database, and at least one medical ontology data source. In a second example of the computer-readable storage medium optionally including the first example, the program is further configured to cause the computer processor to train knowledge graph embeddings based on the knowledge graph, receive new patient data, and generate the healthcare recommendation based on the knowledge graph embeddings and the new patient data. In a third example of the computer-readable storage medium optionally including one or more of the first and second examples, the program is further configured to cause the computer processor to update the knowledge graph with user feedback and user behavior regarding the healthcare recommendations, and rank subsequent healthcare recommendations based on the updated knowledge graph. In a fourth example of the computer-readable storage medium optionally including one or more of the first through third examples, the healthcare insights include one or more of newly-identified edges between entities in the knowledge graph, including one or more of a relationship between a patient and a medication, a relationship between a symptom and a diagnostic code, and a relationship between a healthcare provider and a disease. In a fifth example of the computer-readable storage medium optionally including one or more of the first through fourth examples, the program is further configured to cause the computer processor to update the knowledge graph to include entities relating plain language terminology to medical terminology, and perform a search responsive to a user query including the plain language terminology for one or more of healthcare providers and symptoms with the knowledge graph.

In yet another embodiment, a system comprises a user device configured for a user, and a server communicatively coupled to the client device, the server configured with executable instructions in non-transitory memory of the server that when executed cause a processor of the server to: construct a knowledge graph with data from a heterogeneous plurality of data sources; generate healthcare insights from the knowledge graph; and output, to the user device for display to the user, a healthcare recommendation based on the healthcare insights.

In a first example of the system, the heterogeneous plurality of data sources includes a provider data source, a patient data source, a medicine data source, a disease database, and at least one medical ontology data source, the heterogeneous plurality of data sources communicatively coupled to the server via a network. In a second example of the system optionally including the first example, the server is further configured with executable instructions in non-transitory memory of the server that when executed cause a processor of the server to train knowledge graph embeddings based on the knowledge graph, receive new patient data, and generate the healthcare recommendation based on the knowledge graph embeddings and the new patient data. In a third example of the system optionally including one or more of the first and second examples, the server is further configured with executable instructions in non-transitory memory of the server that when executed cause a processor of the server to update the knowledge graph with user feedback and user behavior regarding the healthcare recommendations, and rank subsequent healthcare recommendations based on the updated knowledge graph. In a fourth example of the system optionally including one or more of the first through third examples, the healthcare insights include one or more of newly-identified edges between entities in the knowledge graph, including one or more of a relationship between a patient and a medication, a relationship between a symptom and a diagnostic code, and a relationship between a healthcare provider and a disease. In a fifth example of the system optionally including one or more of the first through fourth examples, the server is further configured with executable instructions in non-transitory memory of the server that when executed cause a processor of the server to update the knowledge graph to include entities relating plain language terminology to medical terminology, and perform a search responsive to a user query including the plain language terminology for one or more of healthcare providers and symptoms with the knowledge graph. In a sixth example of the system optionally including one or more of the first through fifth examples, the server is further configured with executable instructions in non-transitory memory of the server that when executed cause a processor of the server to transmit search results of the search obtained based on the knowledge graph to the user device for display to the user in a graphical user interface. In a seventh example of the system optionally including one or more of the first through sixth examples, the server is further configured with executable instructions in non-transitory memory of the server that when executed cause a processor of the server to iteratively update the knowledge graph with new and additional data from the heterogeneous plurality of data sources.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
constructing, with a processor, a knowledge graph with data from a heterogeneous plurality of data sources;
generating, with the processor, healthcare insights from the knowledge graph; and
outputting, to a user device for display to a user, a healthcare recommendation based on the healthcare insights.

2. The method of claim 1, wherein the heterogeneous plurality of data sources includes a provider data source, a patient data source, a medicine data source, a disease database, and at least one medical ontology data source.

3. The method of claim 1, further comprising training knowledge graph embeddings based on the knowledge graph, receiving new patient data, and generating the healthcare recommendation based on the knowledge graph embeddings and the new patient data.

4. The method of claim 1, further comprising updating the knowledge graph with user feedback and user behavior regarding the healthcare recommendations, and ranking subsequent healthcare recommendations based on the updated knowledge graph.

5. The method of claim 1, wherein the healthcare insights include one or more of newly-identified edges between entities in the knowledge graph, including one or more of a relationship between a patient and a medication, a relationship between a symptom and a diagnostic code, and a relationship between a healthcare provider and a disease.

6. The method of claim 1, further comprising updating the knowledge graph to include entities relating plain language terminology to medical terminology, and performing a search responsive to a user query including the plain language terminology for one or more of healthcare providers and symptoms with the knowledge graph.

7. A computer-readable storage medium including an executable program stored thereon, the program configured to cause a computer processor to:
Construct a knowledge graph with data from a heterogeneous plurality of data sources;
generate healthcare insights from the knowledge graph; and
output, to a user device for display to a user, a healthcare recommendation based on the healthcare insights.

8. The computer-readable storage medium of claim 7, wherein the heterogeneous plurality of data sources includes a provider data source, a patient data source, a medicine data source, a disease database, and at least one medical ontology data source.

9. The computer-readable storage medium of claim 7, wherein the program is further configured to cause the computer processor to train knowledge graph embeddings based on the knowledge graph, receive new patient data, and generate the healthcare recommendation based on the knowledge graph embeddings and the new patient data.

10. The computer-readable storage medium of claim 7, wherein the program is further configured to cause the computer processor to update the knowledge graph with user feedback and user behavior regarding the healthcare recommendations, and rank subsequent healthcare recommendations based on the updated knowledge graph.

11. The computer-readable storage medium of claim 7, wherein the healthcare insights include one or more of newly-identified edges between entities in the knowledge graph, including one or more of a relationship between a patient and a medication, a relationship between a symptom and a diagnostic code, and a relationship between a healthcare provider and a disease.

12. The computer-readable storage medium of claim 7, wherein the program is further configured to cause the computer processor to update the knowledge graph to include entities relating plain language terminology to medical terminology, and perform a search responsive to a user query including the plain language terminology for one or more of healthcare providers and symptoms with the knowledge graph.

13. A system, comprising:
a user device configured for a user; and
a server communicatively coupled to the client device, the server configured with executable instructions in non-transitory memory of the server that when executed cause a processor of the server to:
construct a knowledge graph with data from a heterogeneous plurality of data sources;
generate healthcare insights from the knowledge graph; and
output, to the user device for display to the user, a healthcare recommendation based on the healthcare insights.

14. The system of claim 13, wherein the heterogeneous plurality of data sources includes a provider data source, a patient data source, a medicine data source, a disease database, and at least one medical ontology data source, the heterogeneous plurality of data sources communicatively coupled to the server via a network.

15. The system of claim 13, wherein the server is further configured with executable instructions in non-transitory memory of the server that when executed cause a processor of the server to train knowledge graph embeddings based on the knowledge graph, receive new patient data, and generate the healthcare recommendation based on the knowledge graph embeddings and the new patient data.

16. The system of claim 13, wherein the server is further configured with executable instructions in non-transitory memory of the server that when executed cause a processor of the server to update the knowledge graph with user feedback and user behavior regarding the healthcare recommendations, and rank subsequent healthcare recommendations based on the updated knowledge graph.

17. The system of claim 13, wherein the healthcare insights include one or more of newly-identified edges between entities in the knowledge graph, including one or more of a relationship between a patient and a medication, a relationship between a symptom and a diagnostic code, and a relationship between a healthcare provider and a disease.

18. The system of claim 13, wherein the server is further configured with executable instructions in non-transitory memory of the server that when executed cause a processor of the server to update the knowledge graph to include entities relating plain language terminology to medical terminology, and perform a search responsive to a user query including the plain language terminology for one or more of healthcare providers and symptoms with the knowledge graph.

19. The system of claim 18, wherein the server is further configured with executable instructions in non-transitory memory of the server that when executed cause a processor of the server to transmit search results of the search obtained based on the knowledge graph to the user device for display to the user in a graphical user interface.

20. The system of claim 13, wherein the server is further configured with executable instructions in non-transitory memory of the server that when executed cause a processor of the server to iteratively update the knowledge graph with new and additional data from the heterogeneous plurality of data sources.
